(19) United States
(12) Patent Application Publication
(10) Pub. No.: US 2008/0125405 A1
(43) Pub. Date: May 29, 2008

(54) Composition for treating or preventing olfactory disorder

(75) Inventors: Hyo-Yul Kim, Seoul (KR); Hun-Jong Dhong, Seoul (KR); Seung-Kyu Chung, Seoul (KR); Kon Chu, Seoul (KR)

Correspondence Address: EDWARDS ANGELL PALMER & DODGE LLP, P.O. BOX 55874, BOSTON, MA, 02205, US

(73) Assignee: Samsung Life Public Welfare Foundation Samsung Medical Center, Seoul (KR)

(21) Appl. No.: 11/823,508

(22) Filed: Jun. 28, 2007

(30) Foreign Application Priority Data
Nov. 29, 2006 (KR) 10-2006-0119152

(51) Int. Cl. A61K 31/56; A61K 31/40; A61P 27/00

(52) U.S. Cl. 514/171; 514/423

(57) ABSTRACT

Disclosed is a composition for treating olfactory disorders, the composition comprising at least one statin-like drug, which is an HMG-CoA reductase inhibitor, as an active ingredient. The composition has excellent effects of protecting and regenerating olfactory nerve.

### CROSS-REFERENCE TO RELATED APPLICATION

The present application claims, under 35 U.S.C. § 119(a), the benefit of the filing date of Korean Patent Application No. 10-2006-0119152 filed on Nov. 29, 2006, the entire contents of which are hereby incorporated by reference.

### BACKGROUND OF THE INVENTION

1. Technical Field

The present invention relates to a composition for treating or preventing olfactory disorders, which comprises a 3-β-hydroxymethylglutarate CoA (HMG-CoA) reductase inhibitor as an active ingredient.

2. Background Art

Olfactory sense is one of the important senses of humans. A person suffering from an olfactory disorder cannot smell foods, enjoy eating, appropriately react to a change in surroundings such as harmful gases or rotten foods, thereby facing lowered quality of life.

As a matter of fact, changes in olfactory sensual functions have been recognized as a problem merely in the field of otorhinolaryngology; other physicians or patients have not focused their attention on such changes.

According to the economic development and extension of medical insurance in recent years, however, there has been an increasing interest in olfactory diseases. Rapid industrialization and modernization has exposed people to pollutants or chemicals capable of functioning as olfactory toxins. Morbidity of olfactory disorders has increased more and more. According to a study in Northern Europe, olfactory diseases are observed in 19% of the total population. Upper respiratory tract infections, aging, diabetes, smoking, or the like are known to cause such olfactory diseases. In addition, diabetes, Alzheimer's diseases, etc. are known to be able to cause olfactory disorders.

Many methods for treating or preventing olfactory diseases have been suggested. One of the methods is to use steroids, vitamins, strychinines, zinc or aminophyllines. Currently, steroids have been widely used. However, steroids are effective only for obstructive olfactory diseases caused by chronic rhinosinusitis or nasal polyp; steroids are not significantly effective for the other olfactory disorders.

Thus, there is still a need for a new agent or method for treating or preventing olfactory disorders.

The information disclosed in this Background of the Invention section is only for enhancement of understanding of the background of the invention and should not be taken as an acknowledgement or any form of suggestion that this information forms the prior art that is already known to a person skilled in the art.

### SUMMARY OF THE INVENTION

The present invention has been made in an effort to solve the above-mentioned problems. It is an object of the present invention to provide a composition and a method for treating or preventing olfactory disorders, which can enhance regeneration of olfactory mucous membranes and induce regeneration of the olfactory nerve, thereby inducing regeneration of olfactory cells.

In one aspect, the present invention provides a composition for treating or preventing olfactory disorders, which comprises 3-β-hydroxymethylglutarate CoA (HMG-CoA) reductase inhibitor as an active ingredient.

Preferably, the HMG-CoA reductase inhibitor is at least one selected from the group consisting of cilastatin, nystatin, lovastatin, somatostatin, pravastatin, simvastatin, fluvastatin, atorvastatin, cervastatin, ulinastatin, rosuvastatin and salts thereof.

In a preferred embodiment, the composition may further comprise a steroid. In this embodiment, suitably, the HMG-CoA reductase inhibitor and the steroid has a weight ratio of 1:0.1 to 1:100.

A preferred composition of the present invention may further comprise at least one additive selected from the group consisting of pharmaceutically acceptable excipients, disintegrating agents, binders and lubricants.

In another aspect, the present invention provides a method for treating or preventing olfactory disorders, which comprises administering to a patient a therapeutically effective amount of 3-β-hydroxymethylglutarate CoA (HMG-CoA) reductase inhibitor.

Preferably, the HMG-CoA reductase inhibitor is at least one selected from the group consisting of cilastatin, nystatin, lovastatin, somatostatin, pravastatin, simvastatin, fluvastatin, atorvastatin, cervastatin, ulinastatin, rosuvastatin and salts thereof.

In a preferred embodiment, the HMG-CoA reductase inhibitor can be administered alone or in combination with a steroid. When the HMG-CoA reductase inhibitor and the steroid are administered, they can be administered together or separately. Suitably, administration by injection, oral injection or transdermal injection can be used.

Preferably, the HMG-CoA reductase inhibitor and the steroid has a weight ratio of 1:0.1 to 1:100.

Also preferably, the administration is made with a daily dose of 0.001-100 mg/kg on the basis of the HMG-CoA reductase inhibitor.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a photographic view showing the results obtained after measuring columnar arrangements corresponding to the arrangements of olfactory epithelial cells of test animals so as to provide grades for evaluation according to a preferred embodiment of the present invention;

FIG. 2 is a graph showing the height of olfactory epithelial cells measured in each test group according to a preferred embodiment of the present invention;

FIG. 3 is a graph showing grades of columnar arrangements corresponding to the arrangements of olfactory epithelial cells in each of the test groups;

FIG. 4 is a photographic view showing tissue specimens of test animals immuno-stained with PGP 9.5 so as to provide grades for evaluation according to a preferred embodiment of the present invention; and

FIG. 5 is a graph showing the immuno-staining degrees in each of the test groups measured after carrying out immuno-staining with PGP 9.5 according to a preferred embodiment of the present invention.

### DETAILED DESCRIPTIONS

HMG-CoA reductase inhibitor (3-β-hydroxymethyl glutarate CoA reductase inhibitor) is a known agent for treating hyperlipidemia. The term “HMG-CoA reductase inhibitor” used herein includes all statin-like drugs.

The statin-like drug used as an active ingredient of the composition according to the present invention includes, but is not limited to, at least one selected from the group consisting of cilastatin, nystatin, lovastatin, somatostatin, pravastatin, simvastatin, fluvastatin, atorvastatin, cervastatin, ulinastatin, rosuvastatin and salts thereof.

The composition according to the present invention may further comprise a steroid. The HMG-CoA reductase inhibitor and the steroid can be used in a weight ratio (HMG-CoA reductase inhibitor:steroid) preferably of 1:0.1 to 1:100, and more preferably of 1:0.5 to 1:50. If the weight ratio is less than 1:0.1, it is not possible to obtain any additional effects provided by the addition of the steroid. If the weight ratio is greater than 1:100, it is not possible to obtain the effects unique to the HMG-CoA reductase inhibitor.

The composition according to the present invention may be effective for treating or preventing various olfactory disorders.

As used herein, the term “olfactory disorders” means a loss or impairment of the olfactory sense and includes damage to the olfactory center or the nervous paths thereof and disorders in the nasal cavity. Main causes of the olfactory disorders include paranasal sinusitis diseases, such as empyema, common colds, allergic rhinitis, loss of olfactory sensory cells caused by head injury, exposure to toxic materials including drugs or industrially harmful substances, diabetes, hormone abnormality, Alzheimer's disease, Parkinson's disease, brain tumor, postoperative complications in the paranasal sinus, or the like.

The olfactory disorders may be classified into conductive or respiratory olfactory disorders, sensorineural olfactory disorders, mixed olfactory disorders and central olfactory disorders. Most common olfactory disorders are sensorineural and mixed olfactory disorders. Sensorineural olfactory disorders include conditions caused by abnormality in mucous membranes or neurons at an olfactory functioning site. Mixed olfactory disorders include conditions of conductive olfactory disorders mixed with sensorineural olfactory disorders, the conductive olfactory disorders including the condition in which air containing a scent cannot be in contact with the olfactory nerve due to nasal diseases, rhinitis occurring after a common cold or ededma in the intranasal mucous membranes.

Such olfactory disorders mean all pathological conditions including impairment in olfactory functions caused by a drop or loss in olfactory cells, and particular examples thereof include anosmia, olfactory hypoesthesia (hyposmia), olfactory hyperesthesia, dysosmia, merosmia, olfactory illusion, or the like.

Anosmia refers to a disease including a loss of the ability to smell; olfactory hypoesthesia refers to a disease consisting of abnormally decreased ability to smell, i.e. ability to smell a strong scent and a loss of ability to smell a weak scent; olfactory hyperesthesia refers to a disease consisting of abnormally increased ability to smell; dysomia refers to a disease including impairment of the sense of smell (a patient suffering from dysomia senses a certain smell as another smell different therefrom); merosmia refers to a disease including a loss of ability to smell a certain scent; and olfactory illusion refers to a disease including conditions in which patients complain of a scent even in the absence of any substances having a scent.

The composition according to the present invention enhances regeneration of olfactory cells so as to treat all kinds of olfactory disorders, particularly anosmia and olfactory hypoesthesia. As mentioned above, anosmia refers to the lack of olfaction, i.e. a loss of the ability to smell. More particularly, anosmia may result from: obstruction of the nasal cavity and a failure in contact between the air and the posterior part in which the olfactory nerve exists due to acute or chronic rhinitis; abnormality in the nasal septum or neoplasm, such as nasal polyp; and abnormality in the posterior part caused by tuberculosis or tumors; and abnormality/loss of central olfactory functions caused by brain tumors or hysteria.

As explained above, olfactory hypoesthesia (hyposmia) refers to a disease consisting of an abnormally decreased ability to recognize stimuli in the olfactory sensory organ, i.e. ability to smell a strong scent and a loss of ability to smell a weak scent, and may results from acute or chronic rhinitis or a common cold as in the case of anosmia.

Preferably, the composition according to the present invention is administered in a controlled amount controlled depending on the particular use and purpose thereof, conditions, age, sex and body weight of a patient, type and severity of patient's disease, administration route and period, or the like. An effective amount of the composition according to the present invention preferably ranges from 0.1 μM to 50 μM as expressed by the concentration of HMG-CoA reductase inhibitor in the blood plasma. The amount of the composition administered actually to achieve the above concentration may depend on the bioavailability of the corresponding formulation of the composition. The composition may be administered in a daily dose ranging from 0.01 to 100 mg/kg (body weight) per day on the basis of HMG-CoA reductase inhibitor, preferably from 0.05 to 50 mg/kg (body weight) per day, and more preferably from 1 to 30 mg/kg (body weight) per day.

The composition according to the present invention may be administered in a single dose or multiple doses per day. Additionally, the composition according to the present invention may be administered via any suitable administration routes including injection, intranasal, parenteral, transdermal, subcutaneous or intradermal administration, oral administration being preferred.

The composition according to the present invention may be formulated into injection formulations, oral formulations, external formulations or inhalation formulations, oral formulations being preferred. Particular examples of the oral formulations include powder, tablets, capsules or liquids. Particular examples of the injection formulations include intravenous injection formulations or intramuscular injection formulations. Particular examples of the external formulations include transdermal absorptive formulations, lotions, emulsions, suspensions, patches, creams or cataplasma formulations. Particular examples of the inhalation formulations include oral inhalation formulations or nasal inhalation formulations. However, pharmaceutical formulations that may be applied to the present invention are not limited thereto.

Additionally, the composition according to the present invention may further comprise, in addition to the active ingredient, additives including pharmaceutically acceptable carriers or diluents, such as conventional excipients, disintegrating agents, binders or lubricants. However, additives that may be used in the present invention are not limited thereto.

The pharmaceutically acceptable carriers or diluents include those currently used in the field of pharmaceutics and non-reactive to the active compound of the present invention. Particular examples of the pharmaceutically acceptable carriers or diluents include: starch, such as corn starch, modified corn starch or potato starch; cellulose derivatives, such as lactose, mannitol, sorbitol, purified white sugar, wood cellulose or microcrystalline cellulose; or inorganic salts, such as croscarmellose sodium, purified gelatin, gum arabic, povidone, magnesium stearate, light anhydrous silicic acid, talc or calcium carbonate.

In a preferred embodiment of the formulation according to the present invention, tablets may be coated with a coating agent, such as hydroxypropylmethyl cellulose, hydroxypropylmethyl cellulose phthalate, cellulose phthalate acetate, titanium oxide, polysorbate or purified white sugar, via a conventional coating process.

In another preferred embodiment of the formulation according to the present invention, external formulations may be combined suitably with liquid oil and fats, solid oil and fats, bees wax, hydrocarbons, higher fatty acids, higher alcohols, esters, surfactants, moisturizing agents, water-soluble polymer compounds, thickening agents, coating agents, lower alcohols, polyhydric alcohols, saccharides, amino acids, organic amines, pH modifiers, anti-oxidants, perfumes or water, as necessary. The above components may also be used in combination.

In still another embodiment of the formulation according to the present invention, patch formulations may include an adhesive substrate including a polymer substrate, such as an acrylic copolymer, polyvinyl pyrrolidone or polyisobutylene, and a plasticizer, such as triethyl citrate, triethylacetyl citrate, glycerin, propylene glycol or polyethylene glycol.

In yet another embodiment of the formulation according to the present invention, injection formulations may be obtained by dissolving the composition according to the present invention into distilled water for injection. If desired, an isotonic agent, analgesic agent, pH modifier, dissolution aid, buffering agent or a preservative may be added thereto. Also, the injection formulation may be provided in the form of a suspension, which may be obtained by suspending the compound according to the present invention in distilled water for injection or vegetable oil. If desired, a base or suspending agent may be added to the injection formulation. Additionally, the injection formulation may be provided in the form of powder or freeze-dried formulation. Such powder type formulations or freeze-dried injection formulations may be dissolved before use, and an excipient or the like may be further added thereto.

Further, the above formulations may be provided in release-controlled formulations so that a predetermined amount of the drug is discharged at a constant rate to be absorbed into the circulation system of a patient according to the particular administration protocol. Particular examples of such release-controlled formulations include matrix-type or coating film-type tablets, granules, capsules in which the same is encapsulated, or transdermal absorptive agents.

In the case of the composition comprising an HMG-CoA reductase inhibitor along with steroids according to the present invention may be provided in the form of a single unit dose formulation including both drugs, or may be formed into two unit dose formulations individually containing each drug, which, in turn, are administered at the same time. In the case of the above single unit dose formulation including both drugs, both drugs may be present independently from each other.

In one embodiment of the present invention, the tablet may include a multi-layer tablet, and the capsule may include one in which the granules of all the ingredients are encapsulated in an adequate ratio. In the case of the injection formulation, all the ingredients are divided into water-soluble ingredients and water-insoluble ingredients and are placed in separate containers. Then, a solution for injection may be obtained, for example, by injecting distilled water for injection into the containers right before use.

Reference will now be made in detail to the preferred embodiments of the present invention. It is to be understood that the following examples are illustrative only and the present invention is not limited thereto.

### EXAMPLE 1

### Preparation of Olfactory Tissue Samples

### 1-1. Test Animals

As test animals, female white Sprague-Dawley rats (Orient Co., Korea) having a body weight of 100-150 g were used. The rats were apparently healthy and had a clean nasal cavity. The test animals were raised in raising chambers for one week.

Each raising chamber was an acryl box including sawdust at the bottom thereof and having window bars at the top thereof for mounting a water container. Three animals were raised in each raising chamber and 72 test animals in total were raised in 24 raising chambers. The test animals were raised under the conditions including a temperature of 21.5-25.5° C., a relative humidity of 45-55%, and a constant lighting cycle of from 7 a.m. to 7 p.m.

Next, 72 test animals accommodated in the raising chambers for one week were divided into three groups, i.e. a control group to which saline was administered, a reference group to which a steroid was administered, and a test group to which atorvastatin was administered, each group including 24 test animals.

The test animals in the test group and the reference group were subjected to intraperitoneal injection of 150 mg/kg (body weight of each test animal) of 3-methylindole (3-MI) so that their olfactory epithelia were damaged. To the test animals in the test group, 10 mg/kg (body weight of each test animal) of atorvastatin dissolved in 1 cc of saline was orally administered at 10 a.m. each day until their tissues were collected after the treatment with 3-methylindole. To the test animals in the reference group, 10 mg/kg (body weight of each test animal) of a steroid formulation, i.e. prednisolone, dissolved in 1 cc of saline was orally administered according to the same protocol as the test group. Additionally, to the test animals in the control group, the same amount (1 cc) of saline was administered after the olfactory epithelia were damaged, according to the same protocol as the test group.

### 1-2. Collection of Olfactory Tissues

To collect the olfactory tissues of the test group, the reference group and the control group, the test animals of the three groups were sacrificed on the fourth day, the seventh day, the fourteenth day and the twenty eighth day after the treatment with 3-methylindole. Six test animals were sacrificed each time.

Collection of the olfactory tissues was carried out as follows: First, 40 mg/kg of ketamin (Yuhan Corp., Korea) and 5 mg/kg of xylazine (Bayer Korea, Korea) were injected to the test animals via an intraperitoneal route to put them under anesthesia, and the test animals were subjected to thoracic incision. After the thoracic incision, in order to measure the total IgE antibody concentration, the left ventricle of each test animal was perforated, 1-2 ml of the blood was sampled, and then the Bouin's fixative (75 ml of picric acid, 25 ml of 40% formalin and 5 ml of glacial acetic acid per 100 ml) was injected through the perforation to carry out cardiac perfusion.

The cardiac perfusion was performed by ligating the inferior vena cava and the inferior aorta and by incising the left ventricle. Next, an 18G injection needle was fixed to the aorta through the incised left ventricle, and perfusion of 400-500 ml of saline and 500-600 ml of the Bouins's fixative was carried out.

After the perfusion, each test animal was cut at its cervical region by using a decapitator, and the scalp, the mandible and the soft tissues in the head were removed. Then, all the bones were trimmed and removed to such a degree that the spleen was not damaged in order to reduce the decalcification period, and the test animal treated as described above was stored in the Bouin's fixative for 24 hours. After carrying out decalcification of the test animal stored in the Bouin's fixative, the test animal was washed thoroughly with water.

To ensure objective comparison by collecting tissue samples at a predetermined site, incision was performed at the structure having the characteristics of the dorsal aspect in the decalcificated animal, i.e. at the coronal plane in the second palatal ridge, so as to obtain a tissue sample. Then, the tissue obtained as described above was fixed with paraffin.

The tissue fixed with paraffin was pretreated via dehydration using an alcohol so as to remove the paraffin used for fixing the tissue. Such dehydration pretreatment was carried out by dipping the tissue into 95% alcohol used in an amount corresponding to 20 times the volume of the tissue and by exchanging the alcohol each day for a period of 5 days. Next, ethanol was added to the tissue to perform alcohol dehydration. Such alcohol dehydration was performed by treating the tissue free from paraffin with an increasing amount of alcohol from a lower concentration to a higher concentration for 1 hour at each concentration until the tissue had a water content of 3-4%.

After carrying out the alcohol dehydration, the alcohol present in the tissue was removed and the alcohol was substituted with xylene to increase the transparency of the tissue. Such xylene substitution was carried out by repeating a treatment cycle three times continuously, each treatment cycle including dipping the dehydrated tissue into 100% xylene twenty times. After the xylene substitution, the tissue was subjected to paraffin embedding including a step of causing paraffin to infiltrate into the voids in the tissue in order to facilitate microtomy of the tissue.

After carrying out the paraffin embedding, the tissue was trimmed so as to cut the paraffin block, and microtomy of the tissue was performed by using a Multi-cut Rotatory Microtome system (Thermo Shandon Ltd., UK) to a thickness of 4 μm.

### EXAMPLE 2

### Determination of Regeneration Effect

### 2-1. H&E Staining

H&E staining of the tissue obtained from Example 1-2 was performed by treating the paraffin block cut into a thickness of 4 μm via the above trimming and microtomy steps of Example 1-2 with a hematoxylin solution (Zymed, USA) and an eosin solution (eosin Y, Sigma, USA). More particularly, the H&E staining was performed as follows.

The paraffin block cut into a thickness of 4 μm as described in Example 1-2, i.e. the tissue specimen, was deparaffinized by repeating a xylene treatment cycle (dipping the tissue into 100% xylene twenty times) three times, was subjected to rehydration and staining with a hematoxylin solution for 5 minutes, and then was washed thoroughly with flowing water for 15 minutes. The washed tissue specimen was subjected to differential staining in 0.25% eosin solution for 40 seconds. After the differential staining, the tissue specimen was treated with an increasing amount of alcohol from a lower concentration to a higher concentration (i.e. 50%, 70%, 95% and 100% alcohol) for 1 hour at each concentration to further carry out dehydration. The dehydrated tissue specimen was passed through xylene three times and was encapsulated by covering it with a cover slide.

### 2-2. Immunohistological Staining

Immunohistological staining of the tissue obtained from the above Example 1-2 was performed by using the paraffin block cut into a thickness of 4 μm via the above trimming and microtomy steps of Example 1-2. To observe the distribution of the olfactory receptor cells, immunoreactivity to protein gene product 9.5 (PGP 9.5) known to exist specifically in the olfactory nerve cells was investigated.

The immunohistological staining was performed by using a Histostain-Plus kit (Zymed, San Francisco, USA). The paraffin block cut into a thickness of 4 μm, i.e. the tissue specimen was positioned on a slide coated with poly-L-lysine and was subjected to rehydration by using a decreasing amount of alcohol gradually from 100% alcohol to distilled water. The rehydrated tissue specimen was introduced into heated 10 mM citrate buffer (pH 5.0, Sigma, USA) and was further heated in a microwave oven twice, for 5 minutes each time. Next, the heated tissue specimen was removed from the microwave oven, cooled in the 10 mM citrate buffer for 20 minutes, washed with distilled water, allowed to react with 0.05M Tris-HCl buffer (Tris buffered saline, TBS) containing 3% H₂O₂ added thereto for 20 minutes to remove the activity of the endogenous peroxidase, and then was washed with 0.05M Tris-HCl buffered saline three times. After removing the activity of the endogenous peroxidase, the washed tissue specimen was treated with 0.1% Triton X-100 (USB, Ohio, USA) for 15 minutes, and further washed with 0.05M Tris-HCl buffered saline three times. After the treatment with 0.1% Triton X-100, the washed tissue specimen was allowed to react with a blocking reagent (10% normal goat serum) for 30 minutes to inhibit non-specific antigen-antibody reactions.

After inhibiting the non-specific antigen-antibody reactions, the tissue specimen was allowed to react with a primary antibody, PGP 9.5 (protein gene product, monoclonal anti-mouse; NOVO, USA), diluted with Tris-HCl buffered saline containing 0.1% bovine serum albumin (BSA) to a ratio of 1:50, overnight at a temperature of 4° C., and then was washed with 0.05M Tris-HCl buffered saline three times, for 5 minutes each time. After allowing the tissue specimen subjected to the reaction with the primary antibody to further react with a biotin-treated secondary antibody (biotinylated secondary antibody, Zymed, San Francisco, USA) at a temperature of 20° C. for 15 minutes, the tissue specimen was washed with water three times, for 5 minutes each time. Then, the tissue specimen subjected to the reaction with the secondary antibody was allowed to further react with streptavidin-peroxidase for 15 minutes, and was further washed with 0.05M Tris-HCl buffered saline three times, for 5 minutes each time. While observing the tissue specimen subjected to the reaction with streptavidin-peroxidase with a microscope, the tissue specimen was developed with 0.05% 3,3′-diaminobenzidine tetrahydrochloride (DAB), and counter-staining was performed by using a mercury-free hematoxylin solution (Zymed, USA). The tissue specimen subjected to the counter-staining was covered with a cover glass to construct a permanent sample.

The paraffin block, i.e. tissue specimen, subjected to the above H&E staining and the immunohistological staining was observed with an optical microscope. More particularly, the tissue observed herein was obtained from the olfactory mucous membranes of both upper sides of the nasal septum in the surface of the tissue prepared via the incision at the coronal plane in the second palatal ridge so as to ensure the observation at a predetermined site. Regeneration of the olfactory mucous membranes was determined by measuring the thickness and arrangement of the olfactory mucous membrane epithelium. Additionally, regeneration of the olfactory nerve was determined by measuring a staining degree of a protein present specifically in the olfactory nerve cells, i.e. PGP 9.5.

Statistical analysis for the above measurements was performed as follows. The height of the olfactory epithelium of the tissue specimen subjected to H&E staining was measured according to the description in [Anova test with Bonferroni's correction, Statistical Analysis in Pharmaceutics and Health Sciences, Geun Young Yu and Jae Uk Ahn, SPSS Academy]. Additionally, statistical analysis for the arrangement of the olfactory mucous membrane epithelial cells and the staining degree in PGP 9.5 staining was performed according to the description in [Kruskal-Wallis test with Bonferroni's correction, Statistical Analysis in Pharmaceutics and Health Sciences, Geun Young Yu and Jae Uk Ahn, SPSS Academy]. The significance level applied in such statistical analysis was 5%.

With reference to the method for determining whether the olfactory functions are recovered or not, various cognitive tests may be used in the case of a human. However, because the animals are devoid of mental capacity, the height and arrangement of the olfactory epithelium were generally observed to histologically determine regeneration of the olfactory epithelium. Otherwise, immunohistochemical staining was performed to observe whether the olfactory nerve is regenerated or not and to estimate regeneration of the olfactory functions.

Hereinafter, regeneration of the olfactory mucous membranes was determined through the results obtained after observing the height and arrangement of the olfactory epithelium of the test animal. Additionally, regeneration of the olfactory receptor cells was determined through the results obtained after observing the immunohistochemical staining degree, so that regeneration of the olfactory functions was determined.

### 2-3. Determination of Regeneration of Olfactory Mucous Membranes

To observe damage to or recovery of the olfactory mucous membrane epithelia, the heights and arrangements of the epithelia in the test groups were compared to each other after carrying out H&E staining. To perform the comparison of the heights of the olfactory epithelia, the height of the olfactory epithelium in each test group was measured by taking a photograph of the olfactory mucous membrane at both upper sides of the nasal septum at a magnitude of 200×, wherein the average height of the olfactory mucous membrane epithelium was determined by using an Image J Program (NIH). Also, to perform the comparison of the heights of the olfactory epithelia, columnar arrangement corresponding to the arrangement of the olfactory epithelial cells in each test group was determined.

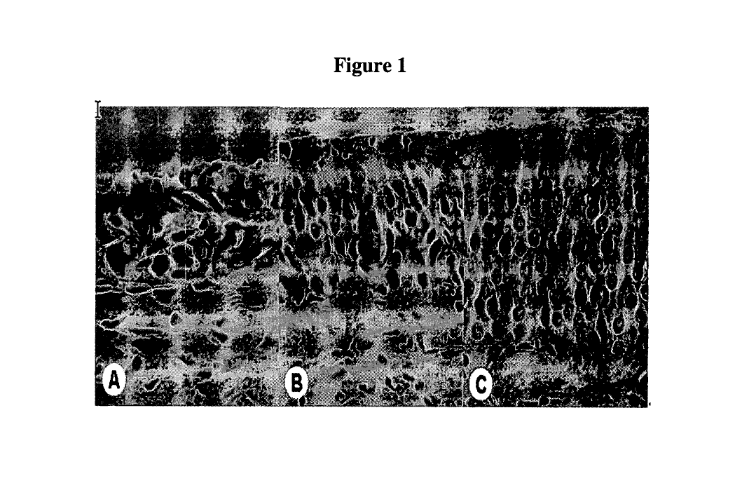

The results are shown in FIG. 1. As shown in FIG. 1, gradation was made according to the following criteria: Grade 1—most of the epithelial cells were not arranged (A in FIG. 1); Grade 2—the epithelial cells were partially arranged (B in FIG. 1); and Grade 3—the epithelial cells were well arranged (C in FIG. 1). Herein, as the grade number increases, a higher degree of regeneration is made in the olfactory mucous membranes.

### 2-3-1. Measurement of Height of Olfactory Epithelium

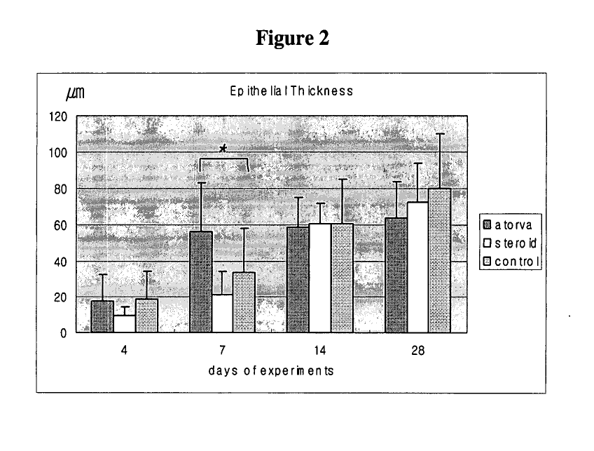

After measuring the heights of the olfactory epithelia, statistical analysis of the results was performed by using the above mentioned Anova test with Bonferroni's correction. The results are shown in FIG. 2.

As shown in FIG. 2, the olfactory epithelium of the control treated with 3-methylindole and administered with saline shows a height of 18.9±15.2 μm, that of the reference group administered with a steroid shows a height of 9.5±4.7 μm, and that of the test group administered with atorvastatin shows a height of 17.4±15.1 μm. The above results indicate that the olfactory epithelium is damaged by the treatment with 3-methyl indole. Additionally, the test group shows a relatively low separation of the olfactory epithelium, but no statistical significance is observed in this test (p=0.165).

Additionally, seven days after the treatment with 3-methylindole, the control group shows a thickness of 33.6±24.4 μm, the reference group shows a thickness of 21.1±13.4 μm, and the test group shows a thickness of 56.1±26.9 μm. Therefore, there is a significant increase in thickness of the olfactory epithelium in the test group, when comparing the test group administered with atorvastatin to the reference group and the control group (p=0.002). Also, in the case of intergroup comparison using the Bonferroni's correction, the test group administered with atorvastatin shows a significant difference as compared to the reference group administered with the steroid (p=0.002).

Then, fourteen days after the treatment with 3-methylindole, the control group shows a thickness of 60.4±24.7 μm, the reference group shows a thickness of 60.5±11.6 μm, and the test group shows a thickness of 58.6±16.3 μm (p=0.963). Further, twenty eight days after the treatment with 3-methylindole, the control group shows a thickness of 80.1±29.8 μm, the reference group shows a thickness of 72.2±21.8 μm, and the test group shows a thickness of 63.9±20.2 μm (p=0.317).

### 2-3-2. Determination of Arrangement of Olfactory Epithelial Cells

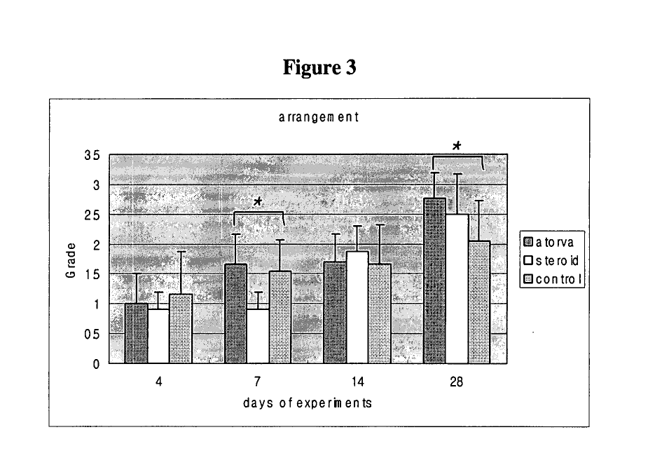

Additionally, columnar arrangement of the olfactory epithelial cells in each group was determined. The columnar arrangement of each group is shown in FIG. 3 as a grade evaluated according to the above criteria.

As shown in FIG. 3, four days after the treatment with 3-methylindole, the control group shows an average grade (corresponding to the columnar arrangement of olfactory epithelial cells) of 1.2±0.7, the reference group administered with the steroid shows an average grade of 0.9±0.3, and the test group administered with atorvastatin of 1.0±0.5, no statistical significance being observed (p=0.517).

Next, seven days after the treatment with 3-methylindole, the control group, the reference group and the test group show an average grade of 1.5±0.41, 0.9±0.5 and 1.7±0.3, respectively, a significant difference being observed among the three groups (p=0.002). Also, in the case of intergroup comparison, a significant difference is observed between the test group and the reference group (p=0.001).

Additionally, fourteen days after the treatment with 3-methylindole, the control group, the reference group and the test group show an average grade of 1.7±0.7, 1.8±0.5 and 1.7±0.7, respectively (p=0.512). Further, twenty eight days after the treatment with 3-methylindole, the control group, the reference group and the test group show an average grade of 2.1±0.7, 2.5±0.4 and 2.8±0.5. The above results indicate that there is a significant difference among the three groups (p=0.037) and between the test group and the control group in terms of intergroup comparison (p=0.030).

### 2-4. Determination of Regeneration of Olfactory Receptor Cells

To determine the regeneration of the olfactory receptor cells, the tissue specimen subjected to immunohistological staining with PGP 9.5 as described in Example 2-2 was photographed, and expression of PGP 9.5 was measured on the resultant photographic image via a semi-quantitative method. To analyze the results obtained from the above measurement, reactivity to PGP 9.5 depending on degrees of damage to or regeneration of the olfactory receptor cells was measured.

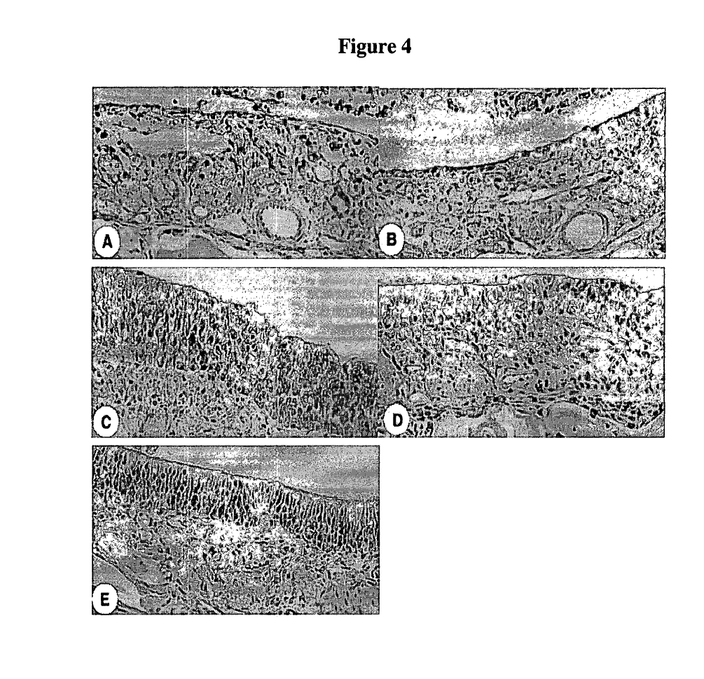

The results are shown in FIG. 4. As shown in FIG. 4, regeneration of the olfactory receptor cells was graded according to the following criteria: Grade 0—no olfactory epithelial cells were observed (A in FIG. 4); Grade 1—olfactory epithelial cells were observed but no cells showing a positive reactivity to PGP 9.5 were observed (B in FIG. 4); Grade 2—only the base cell layer in the olfactory epithelium was stained (C in FIG. 4); Grade 3—arrangement of the olfactory epithelial layer was abnormal, or a half or less of the cells shows a positive reactivity to PGP 9.5 (D in FIG. 4); Grade 4—arrangement of the olfactory epithelial layer was normal and a half or more of the cells shows a positive reactivity to PGP 9.5 (E in FIG. 4). As the grade number increases, a higher degree of regeneration is made in the olfactory receptor cells.

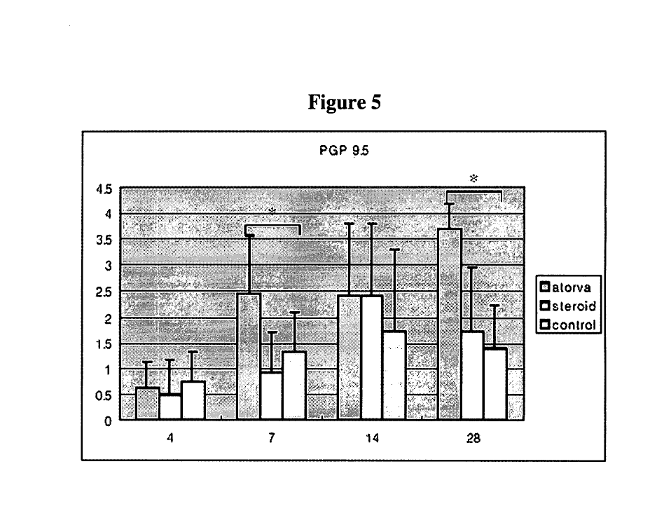

Additionally, reactivity to PGP 9.5 of each of the control group, the reference group and the test group is shown in FIG. 5 as an average PGP 9.5 immunostaining grade evaluated according to the above criteria. Then, statistical analysis of the results as shown in FIG. 5 was performed by using the Kruskal-Wallis test with Bonferroni's correction.

As shown in FIG. 5, four days after the administration of 3-methylindole, the control group, the reference group and the test group show an average PGP 9.5 immunostaining grade (indicating regeneration of the olfactory receptor cells) of 0.75±0.58, 0.5±0.67 and 0.64±0.51, respectively, no statistical significance being observed among the three groups (p=0.461).

Next, seven days after the administration of 3-methylindole, the control group, the reference group and the test group show an average grade of 1.33±0.78, 0.92±0.79 and 2.46±1.12, respectively, a significant difference being observed among the three groups (p=0.003). Also, in the case of intergroup comparison, a significant difference is observed between the test group and the reference group, as well as between the test group and the control group (p=0.001 and 0.014, respectively).

Additionally, fourteen days after the administration of 3-methylindole, the control group, the reference group and the test group show an average grade of 1.75±1.55, 2.42±1.38 and 2.42±1.38, respectively (p=0.360). Further, twenty eight days after the administration of 3-methylindole, the control group, the reference group and the test group show an average grade of 1.4±0.84, 1.75±1.22 and 3.7±0.48. The above results indicate that there is a significant difference among the three groups (p<0.001). In the case of intergroup comparison, it can be seen that the test group shows a significantly higher grade as compared to the reference group and the control group (p=<0.001).

As can be seen from the above test results, at least seven days after the treatment with 3-methylindole, the olfactory mucous membranes damaged by 3-methylindole can be healed according to the present invention, on the basis of the measurements of the height and arrangement of the olfactory epithelium. Also, it can be seen from the above results obtained after staining of the olfactory receptor cells that the olfactory nerve cells can be generated. Further, the above results indicate that the test group treated according to the present invention provides more excellent effects of healing damage to the olfactory mucous membranes and regenerating the olfactory nerve cells, when compared to the control group and the reference group administered with a steroid formulation, i.e. prednisolone.

Although several preferred embodiments of the present invention have been described for illustrative purposes, those skilled in the art will appreciate that various modifications, additions and substitutions are possible, without departing from the scope and spirit of the invention as disclosed in the accompanying claims.

What is claimed is:

1. A composition for treating or preventing olfactory disorders, which comprises 3-β-hydroxymethylglutarate CoA (HMG-CoA) reductase inhibitor as an active ingredient.

2. The composition of claim 1, wherein the HMG-CoA reductase inhibitor is at least one selected from the group consisting of cilastatin, nystatin, lovastatin, somatostatin, pravastatin, simvastatin, fluvastatin, atorvastatin, cervastatin, ulinastatin, rosuvastatin and salts thereof.

3. The composition of claim 1, wherein the HMG-CoA reductase inhibitor is atorvastatin.

4. The composition of claim 1, wherein the olfactory disorder is anosmia or olfactory hyperesthesia.

5. The composition of claim 1 further comprising a steroid.

6. The composition of claim 5, wherein the HMG-CoA reductase inhibitor and the steroid has a weight ratio of 1:0.1 to 1:100.

7. The composition of claim 1 further comprising at least one additive selected from the group consisting of pharmaceutically acceptable excipients, disintegrating agents, binders and lubricants.

8. The composition of claim 5 further comprising at least one additive selected from the group consisting of pharmaceutically acceptable excipients, disintegrating agents, binders and lubricants.

9. A method for treating or preventing olfactory disorders, which comprises administering to a patient a therapeutically effective amount of 3-β-hydroxymethylglutarate CoA (HMG-CoA) reductase inhibitor.

10. The method of claim 9, wherein the HMG-CoA reductase inhibitor is at least one selected from the group consisting of cilastatin, nystatin, lovastatin, somatostatin, pravastatin, simvastatin, fluvastatin, atorvastatin, cervastatin, ulinastatin, rosuvastatin and salts thereof.

11. The method of claim 9, wherein the HMG-CoA reductase inhibitor is atorvastatin.

12. The method of claim 9, wherein the olfactory disorder is anosmia or olfactory hyperesthesia.

13. The method of claim 9, wherein the administration is made by injection, orally or transdermally.

14. The method of claim 9, further comprising administering a steroid.

15. The method of claim 14, wherein the HMG-CoA reductase inhibitor and the steroid are administered together or separately.

16. The method of claim 15, wherein the HMG-CoA reductase inhibitor and the steroid has a weight ratio of 1:0.1 to 1:100.

17. The method of claim 14, wherein the administration is made by injection, orally or transdermally.

18. The method of claim 9, wherein the administration is made with a daily dose of 0.001-100 mg/kg on the basis of the HMG-CoA reductase inhibitor.
